(19) United States
(12) Patent Application Publication
(10) Pub. No.: US 2004/0048289 A1
(43) Pub. Date: Mar. 11, 2004

(54) Magnetic particle membrane-specific protein

(76) Inventors: Takashi Matsunaga, Tokyo (JP); Haruko Takeyama, Tokyo (JP); Yoshiko Okamura, Tokyo (JP)

Correspondence Address: Morgan & Finnegan, Suite 400, 1775 Eye Street, Washington, DC, 20006, US

(21) Appl. No.: 10/450,346

(22) PCT Filed: Oct. 11, 2001
(86) PCT No.: PCT/JP01/08918

(30) Foreign Application Priority Data
Dec. 14, 2000 (JP) 2000-381015

(51) Int. Cl. C12Q 1/68; C07H 21/04; C12N 1/20; C07K 14/195

(52) U.S. Cl. 435/6; 435/69.1; 435/252.3; 435/320.1; 530/400; 536/23.7

(57) ABSTRACT

The object of the present invention is to provide Mms 16, a protein specific to a magnetic particle membrane derived from a magnetic bacterium (Magnetospirillum sp.) AMB-1, DNA which encodes the protein, and a sandwich immunoassay method and a pharmaceutical composition, etc., using the same. Mms 16, a novel protein specific to a bacterial magnetic particle membrane, was identified by: fractionating cell homogenate of magnetic bacteria into 3 fractions of cell membranes, magnetic particle membranes, and cytoplasms, and by comparing the SDS-PAGE profiles of each protein. Further, an amino acid sequence of the protein or a base sequence of a gene that encodes the protein was determined and it was confirmed that the protein exhibited GTPase activity.

### TECHNICAL FIELD

The present invention relates to Mms 16, a protein specific to a magnetic particle membrane, DNA which encodes the protein, and their use.

### BACKGROUND ART

In a bacterial component, a magnetic bacterial-particle produced by a magnetic bacterium has a structure called magnetosome where 10 to 20 magnetic particles of 50 to 100 nm in particle diameter are concatenated, and is covered with an organic membrane about 2 to 4 nm thick. Configuration of bacterial magnetic particles synthesized by magnetic bacteria includes octahedron, hexagonal prism and bullet, and these conformations are observed to be species-specific (Appl. Microbiol. Biotechnol. 52, 464-73, 1999). This fact strongly suggests that there is a species-specific controlling mechanism of crystal conformation in a magnetic bacterium, and it is thought that a membrane protein on a bacterial magnetic particle membrane is involved in the controlling mechanism (Adv. Microbiol. Biotechnol. 31, 125-181, 1990). Further, for the formation of magnetites in vesicles, a protein specific to magnetosome is expected to have specific functions such as accumulation of iron ion, formation of crystal nucleus, reduction of minerals and pH control (Adv. Microbiol. Biotechnol. 31, 125-181, 1990; J. Bacteriol. 170, 834-841, 1988).

With regard to proteins on a magnetic particle membrane, Gorby et al. (J. Bacteriol. 170, 834-841, 1988) have so far observed proteins of 15, 33 kDa specific to a magnetic particle membrane in an MS-1 strain based on comparison of profiles of proteins, being obtained by SDS-PAGE, of cell membranes, magnetic particle membranes, or cytoplasms. In order to fractionate a low molecular weight protein in an MS-1 strain by the same method, Okuda et al. (Gene 171, 99-102, 1996) conducted Tricine/SDS-PAGE. As a result, proteins of 12, 22, 28 kDa have been revealed to be specific to a magnetic particle membrane. The present inventors have also identified specific proteins of 24.8, 35.6, 66.0 kDa in an AMB-1 strain (Biochem. Biophys. Res. Commun. 268, 932-937, 2000). Among these proteins specific to a bacterial magnetic particle membrane, mam 22, a gene of a 22 kDa protein (Gene 171, 99-102, 1996), and mps A, a gene of a 35.6 kDa protein (Biochem. Biophys. Res. Commun. 268, 932-937, 2000) were cloned and their functions were estimated on the basis of motif analysis, however, functional analysis has not been conducted and there are no further reports about other proteins. In addition, it is reported that mag A, a gene related to the production of a magnetic particle which had been separated by the present inventors, encodes an iron-transport protein and localizes on a cell membrane and a bacterial magnetic particle membrane (J. Biol. Chem. 270, 28392-28396, 1995; J. Biochem. 118, 23-27, 1995). This Mag A protein is the only protein on a bacterial magnetic particle membrane that has been functionally analyzed so far.

Some bacteria maintain intracellular membrane structures for specialized metabolism such as photosynthesis, nitrification, oxidization of methane and nitrogen fixation. However, it has not been reported that a bacterium which maintains an intracellular membrane structure system capable of containing a material is observed. Though it is observed that said metabolism-related membrane structure is derived from a cell membrane, its forming mechanism has not been reported and it is unknown where a bacterial magnetic particle membrane derived from. Recently, the present inventors have reported 5 proteins of 12.0, 16.0, 24.8, 35.6 (Mps A) and 66.2 kDa which specifically express on a bacterial magnetic particle membrane of a magnetic bacterium (Magnetospirillum sp.) AMB-1 (ATCC700264) (Appl. Biochem. Biotech. 84-86, 441-446, 2000) and also have reported that a protein of 16.0 kDa, which exhibits most abundant expression on a bacterial magnetic particle membrane, separates into two spots by two-dimensional electrophoresis, but the details have been unknown.

On the other hand, in Japanese Laid-Open Patent Application No. 5-209884, there is a disclosure of a method which can precisely detect extremely small quantities of antigens/antibodies comprising the steps of: a fluorescence-labeled antibody or antigen is fixed to a bacterial magnetic particle extracted from a magnetic bacterium with the use of pyridyldithioalkyl fatty acid/N-succinimidyl ester; antigen-antibody reaction is caused by using an antigen or an antibody to the fixed antibody or antigen to produce an aggregate; the aggregate is magnetically separated and concentrated, and its fluorescence concentration is measured. Further, in Japanese Laid-Open Patent Application No.6-261745, a magnetic bacterium having sulfate reducing ability that is useful for treatment of sewage with use of activated sludge, and for synthesis of magnetite ultrafine particles with uniform shape and size is disclosed, and Japanese Laid-Open Patent Application No. 7-241192 discloses a liposome that contains a magnetosome and a gene of a magnetic bacterium, and a method for introducing a gene into a cell comprising a step of directing the liposome to a cell and contacting the liposome with the cell by applying a magnetic field to the liposome.

The object of the present invention is to provide Mms 16, a protein specific to a magnetic particle membrane derived from a magnetic bacterium (Magnetospirillum sp.) AMB-1 (16 kDa protein), and DNA which encodes the protein, and a sandwich immunoassay method and pharmaceuticals using the same, etc.

### DISCLOSURE OF THE INVENTION

The inventors of the present invention have conducted intensive study to attain the above-mentioned object, and fractionated the cell homogenate of the magnetic bacterium into 3 fractions of cell membranes, magnetic particle membranes and cytoplasms, compared the SDS-PAGE profiles of each protein, identified Mms16, a novel protein specific to a bacterial magnetic particle membrane, performed sequence determination of an amino acid sequence of the protein and a base sequence of a gene that encodes the protein. As a result, it has been found that N-terminal partial sequence of the protein is homologous to GTP binding protein and is GTPase having GTP binding ability, and the present invention has thus completed.

The present invention relates to DNA that encodes a protein described in the following (a) or (b): (a) a protein comprising an amino acid sequence represented by Seq. ID No. 2, (b) a protein comprising an amino acid sequence wherein one or a few amino acids are deficient, substituted or added in an amino acid sequence represented by Seq. ID No. 2, and having GTPase activity (claim 1), DNA that contains a base sequence represented by Seq. ID No. 1, its complementary sequence, and part or whole of these sequences (claim 2), DNA that encodes a protein hybridizing with DNA according to claim 2 under stringent conditions and having GTPase activity (claim 3), a promoter having high transcriptional activity that comprises part of DNA according to claim 2 or 3 (claim 4), a protein comprising an amino acid sequence represented by Seq. ID No. 2 (claim 5), a protein comprising an amino acid sequence wherein one or a few amino acids are deficient, substituted or added in an amino acid sequence represented by Seq. ID No.2, and having GTPase activity (claim 6), and a peptide comprising part of the protein according to claim 5 or 6, and having GTPase activity (claim 7).

The present invention also relates to a fusion protein or a fusion peptide wherein the protein according to claim 5 or 6, or the peptide according to claim 7 is bound to a functional protein or a functional peptide (claim 8), the fusion protein or the fusion peptide according to claim 8, wherein the functional peptide is part of a functional protein, or a peptide tag (claim 9), the fusion protein or the fusion peptide according to claim 8 or 9, wherein the functional protein is one or more proteins selected from antigen, antibody, receptor, lectin, hormone, binding protein, enzyme, and marker protein (claim 10), an antibody that specifically binds to the protein according to claim 5 or 6, or the peptide according to claim 7 (claim 11), the antibody according to claim 11, wherein the antibody is a monoclonal antibody (claim 12), and a recombinant protein or a peptide that specifically binds to the antibody according to claim 11 or 12, and has GTPase activity (claim 13).

The present invention further relates to a host cell comprising an expression system that can express the protein according to claim 5 or 6, the peptide according to claim 7, or the fusion protein or the fusion peptide according to any of claims 8 to 10 (claim 14), the host cell according to claim 14, wherein the host cell is a host magnetic bacterium, Magnetospirillum sp. (claim 15), the host cell according to claim 15, wherein the host magnetic bacterium, Magnetospirillum sp. is AMB-1 (FERM BP-5458, ATCC700264), MS-1 (IF015272, ATCC31632, DSM3856), MSR-1 (IF015272, DSM6361), RS-1 (FERM BP-13283), or MGT-1 (FERM P-16617) (claim 16), and a magnetic particle that can be obtained from the host magnetic cell according to claim 15 or 16 (claim 17).

The present invention also relates to a sandwich immunoassay method that uses an antibody being expressed on the magnetic particle according to claim 17, and a labeled antibody (claim 18), a magnetic liposome that comprises the magnetic particle according to claim 17 (claim 19), and a pharmaceutical composition that comprises the magnetic liposome according to claim 19 as an active component (claim 20).

### BRIEF EXPLANATION OF DRAWINGS

FIG. 1 is a view showing the amino acid sequence of ORF that encodes Mms 16, a protein specific to a magnetic particle membrane of the present invention, obtained by genomic sequence.

FIG. 2 is a view showing a base sequence of mms 16 gene of the present invention and its adjacent regions.

FIG. 3 is a view showing the results of SDS-PAGE (A) and Western blotting (B) of the Mms 16-hemagglutinin epitope tag fusion protein of the present invention being expressed in E. coli.

FIG. 4 is a view showing the result of studying GTP binding ability of the Mms 16-hemagglutinin epitope tag fusion protein of the present invention being expressed in E. coli.

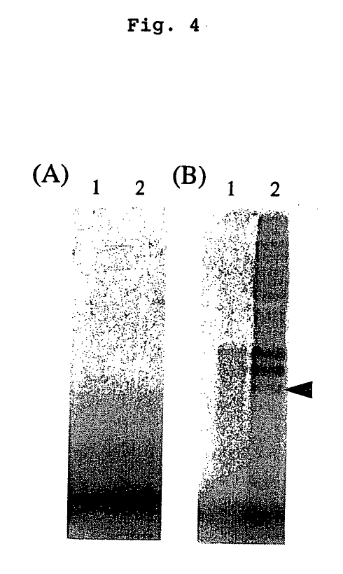

FIG. 5 is a view showing the result of studying GTPase activity of the Mms 16-hemagglutinin epitope tag fusion protein of the present invention being expressed in E. coli.

### BEST MODE OF CARRYING OUT THE INVENTION

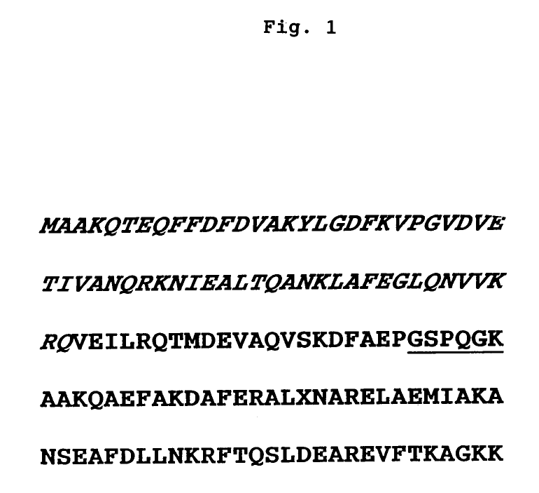

A protein as an object of the present invention includes Mms 16, a protein specific to a magnetic particle membrane, which comprises an amino acid sequence represented by FIG. 1 or Seq. ID No.2 in the sequence listing, and a protein comprising an amino acid sequence wherein one or a few amino acids are deficient, substituted or added in an amino acid sequence represented by Seq. ID No. 2, and having GTPase activity. In FIG. 1, the italics represent a partial sequence obtained by N-terminal amino acid sequence, and the underlined part represents a sequence that conserves ATP/GTP-binding motif (P-loop). As a peptide of the present invention, a peptide comprising part of the protein of the present invention and having GTPase activity is exemplified. The protein and the peptide of the present invention, which comprise the protein and the peptide as an object of the present invention mentioned above, and a recombinant protein and a peptide that are specifically bound by an antibody which specifically bind to the above-mentioned protein and peptide as an object of the present invention, are hereinafter collectively called as “the present protein/peptide”. The present protein/peptide can be prepared, for example, by a method comprising the steps of fractionating cell homogenate of a magnetic bacterium (Magnetospirillum sp.) AMB-1 into 3 fractions (cell membranes, magnetic particle membranes and cell membranes) and comparing the SDS-PAGE profiles of each protein, and a known method based on DNA sequence information etc. of the present protein/peptide, and it is not particularly limited where the present protein/peptide are derived from. The above-mentioned magnetic bacterium (Magnetospirillum sp.) AMB-1 was deposited with the National Institute of Bioscience and Human Technology (Tsukuba, Ibaraki) on Nov. 12, 1992, with the accession no. “FERM BP-5458” under the Budapest Treaty on the international recognition of the deposit of microorganisms for the purpose of patent procedure.

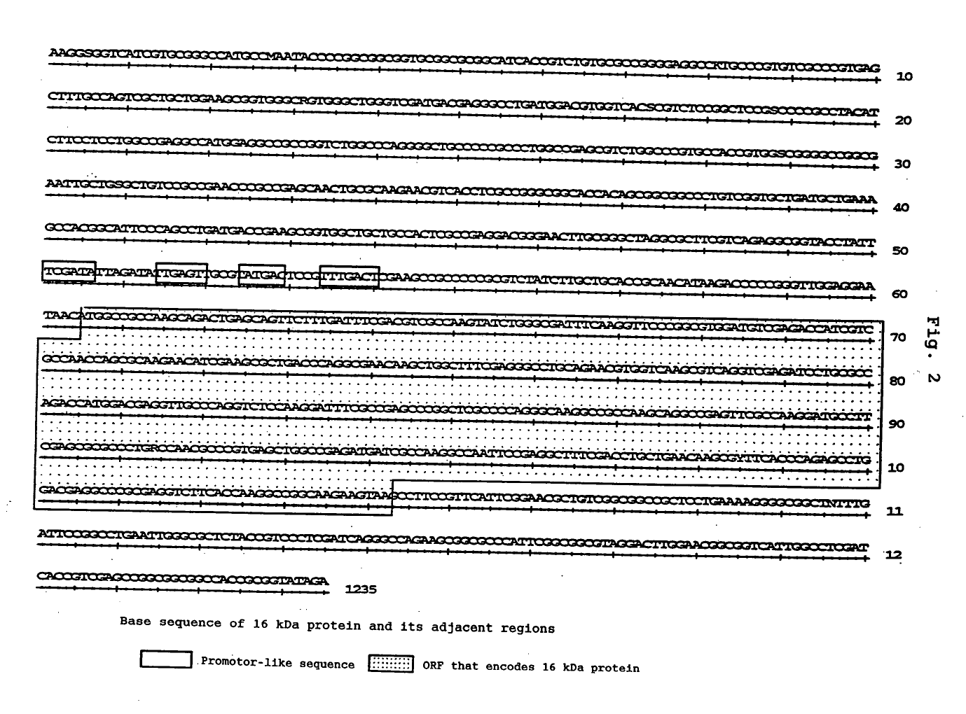

Specific examples of DNA as an object of the present invention are DNA that encodes Mms 16, a protein specific to a magnetic particle membrane, comprising an amino acid sequence represented by Seq. ID No. 2, DNA that encodes a protein comprising an amino acid sequence wherein one or a few amino acids are deficient, substituted or added in an amino acid sequence represented by Seq. ID No. 2, and having GTPase activity, DNA that contains a base sequence represented by Seq. ID No. 1 or its complementary sequence, and part or whole of these sequences. A base sequence of mms 16 gene of the present invention and its adjacent regions is shown in FIG. 2 by way of suggestion. As shown in FIG. 2, part of the base sequence represented by Seq. ID No. 1 comprises a palindrome that can bind to a promoter having high transcriptional activity, and a presumable factor that regulates the condition under which magnetic particles are not produced. These can be prepared by a known method based on their DNA sequence information.

Further, DNA of the interest that encodes a protein having GTPase activity, and has an effect same as that of DNA of Mms 16, a protein specific to a magnetic particle membrane, can be obtained by performing hybridization of various DNA libraries under stringent conditions, with use of a base sequence represented by Seq. ID No. 1, or its complementary sequence, and part or whole of these sequences as a probe, and then isolating DNA that hybridizes with the probe. As a hybridization condition for obtaining the DNA, hybridization at 420 C, and washing at 42° C. by a buffer containing 0.1×SSC, 0.1% SDS are exemplified, and more preferably, hybridization at 65° C., and washing at 65° C. by a buffer containing 1×SSC, 0.1% SDS are exemplified. In addition to the above-mentioned temperature condition, there are various factors that affect the stringency of hybridization, and one skilled in the art can realize the same stringency as the stringency of hybridization exemplified above by combining the various factors appropriately.

As a fusion protein and a fusion peptide of the present invention, it is possible to use any fusion protein and any fusion peptide wherein the present protein/peptide and a functional protein or a functional peptide are bound. Examples of the functional protein include immune-related proteins such as antigen, antibody and protein A, binding proteins that have binding ability such as lectin, avidin and biotin, enzymes such as coenzyme, hydrolase, oxidoreductase, isomerase, transferase, lyase and restriction enzyme, various receptors, marker proteins such as GFP. Examples of the functional peptide include, and without being limited thereto, peptide comprising part of the above-mentioned functional protein, epitope tags such as HA (hemagglutinin), FLAG and Myc, and affinity tags such as GST, maltose-binding protein, biotinylated peptide and oligohistidine. A fusion functional protein wherein one or more functional proteins or functional peptides are fused may also be used. The fusion proteins and the fusion peptides can be produced by usual methods, and are useful for purification of the present protein/peptide using affinity between Ni-NTA and His tag, for detection of the present protein/peptide, and as an investigative reagent in the art.

As an antibody that specifically binds to the above-mentioned proteins or peptides of the present invention, an immune-specific antibody such as monoclonal antibody, polyclonal antibody, chimeric antibody, single-stranded antibody, humanized antibody, etc. are specifically exemplified, and these antibodies can be produced by usual methods using part or whole of Mms 16, the above-mentioned protein specific to a magnetic particle membrane, as an antigen. Among them, however, a monoclonal antibody is more preferable because of its specificity. An antibody that specifically binds to Mms 16, a protein specific to a magnetic particle membrane, such as the monoclonal antibody, is useful, for example, for elucidating the molecular mechanism of a protein specific to a magnetic particle membrane.

The above-mentioned antibody of the present invention is produced by administrating the present protein/peptide or a fragment thereof containing an epitope, or a cell expressing the protein on its membrane surface to (preferably non-human) animals with commonly used protocols. For instance, in order to prepare monoclonal antibodies, any methods that bring antibodies produced by cultures of continuous cell lines, such as hybridoma (Nature 256, 495-497, 1975), trioma, human B cell hybridoma (Immunology Today 4, 72, 1983) and EBV-hybridoma (MONOCLONAL ANTIBODIES AND CANCER THERAPY, pp. 77-96, Alan R. Liss, Inc., 1985) can be used.

In order to develop a single stranded antibody to the above-mentioned present protein/peptide of the present invention, the preparation method of single stranded antibodies (U.S. Pat. No. 4,946,778) can be applied. Further, in order to express a humanized antibody, it is possible to use transgenic mice, other mammalian animals or the like, and to isolate and identify the clones that express the present protein/peptide with the above-mentioned antibodies, and to purify the polypeptide by affinity chromatography. An antibody to the present protein/peptide is useful as various diagnostic drugs and pharmaceuticals, and for elucidating the mechanism of magnetic particle membrane formation. Further, recombinant proteins and peptides to which these antibodies specifically bind are also included in the present protein/peptide of the present invention as mentioned above.

It is possible to analyze the function of the present protein/peptide by using the above-mentioned antibodies such as monoclonal antibodies labeled with, for example, fluorescent materials such as FITC (fluorescein isothiocyanate) or tetramethylrhodamine isothiocyanate; with radioisotopes such as ¹²⁵I, ³²P, ¹⁴C, ³⁵S or ³H; or with enzymes like alkaline phosphatase, peroxidase, β-galactosidase or phycoerythrin; or by using fusion proteins where the above-mentioned antibodies such as monoclonal antibodies are fused with fluorescence emission proteins such as green fluorescent protein (GFP). Examples of an immunoassay using the antibody of the present invention include RIA method, ELISA method, fluorescent antibody technique, plaque method, spot method, hemagglutination reaction method and Ouchterony method.

The present invention also relates to a host cell which contains an expression system that can express the above-mentioned present protein/peptide, a fusion protein or a fusion peptide. The gene that encodes the present protein/peptide can be introduced into a host cell by methods described in a number of standard laboratory manuals, such as, by Davis et al. (BASIC METHODS IN MOLECULAR BIOLOGY, 1986), and by Sambrook et al. (MOLECULAR CLONING: A LABORATORY MANUAL, 2nd Ed., Cold Spring Harbor Laboratory Press, Cold Spring Harbor, N.Y., 1989), etc. Examples of those methods include calcium phosphate transfection, DEAE-dextran-mediated transfection, transvection, microinjection, cationic liposome-mediated transfection, electroporation, transduction, scrape loading, ballistic introduction and infection. Examples of the host cells include bacterial procaryotic cells such as magnetic bacteria, Escherichia coli, Streptomyces, Bacillus subtilis, Streptococcus, Staphylococcus, etc.; fungous cells such as yeast, Aspergillus, etc.; insect cells such as drosophila S2, spodptera Sf 9, etc., and animal cells such as L cells, CHO cells, COS cells, HeLa cells, C127 cells, BALB/c3T3 cells (including mutant strains deficient in dihydrofolate reductase, thymidine kinase or the like), BHK21 cells, HEK293 cells, Bowes melanoma cells, etc., and plant cells. However, magnetic bacteria are preferably used because it is easy to collect them with magnetism. Specific examples of the magnetic bacterium (Magnetospirillum sp.) include, and without being limited thereto, AMB-1 (FERM BP-5458), MS-1 (IF015272, ATCC31632, DSM3856), MSR-1 (IF015272, DSM6361), RS-1 (FERM BP-13283), MGT-1 (FERM P-16617).

As the expression system, any expression system that can express the above-mentioned present protein/peptide, a fusion protein or a fusion peptide in a host cell can be used. Examples of the expression system include expression systems derived from chromosome, episome and virus, for example, vectors derived from bacterial plasmid, yeast plasmid, papovavirus like SV40, vaccinia virus, adenovirus, chicken pox virus, pseudorabies virus, or retrovirus, vectors derived from bacteriophage, transposon, and the combination of these, for instance, vectors such as cosmid or phagemid, being derived from genetic factors of plasmid and of bacteriophage. However, a recombinant plasmid vector having a promoter derived from a magnetic bacterium, such as the promoter having high transcriptional activity of the present invention mentioned above, HT3 promoter (Japanese Laid-Open Patent Application No. 11-243963), mag promoter (Japanese Laid-Open Patent Application No. 8-228782), and mps promoter (WO97/35964), is preferable. These expression systems may contain a regulatory sequence that acts not only as a promoter but also as a controller of expressions.

A host cell such as a host magnetic bacterium that comprises the above-mentioned expression system, and the above-mentioned present protein/peptide or the fusion protein/peptide that can be obtained by culturing the cell, and a magnetic particle expressing the present protein/peptide or the fusion protein/peptide on a magnetic particle membrane, which is obtainable from the above-mentioned host magnetic bacterium, can be used in a sandwich immunoassay method or a magnetic liposome of the present invention as hereinafter described. In order to collect the present protein/peptide from cell cultures and purify it, publicly known methods including electrophoresis, ammonium sulfate or ethanol precipitation, acid extraction, anion or cation exchange chromatography, phosphocellulose chromatography, hydrophobic-interaction chromatography, affinity chromatography, hydroxyapatite chromatography, and lectin chromatography can be used, and electrophoresis is preferably used among them. As columns used for affinity chromatography, for example, there are columns to which an antibody to the present protein/peptide is bound, or in case where a normal peptide tag is added to the present protein/peptide mentioned above, there are columns to which materials having affinity to the peptide tag are bound, and the present protein/peptide can be obtained by using these columns. The method for purifying the present protein/peptide mentioned above can be applied to peptide synthesis as well.

A magnetic particle (magnetosome) expressing the above-mentioned present protein/peptide or the fusion protein/peptide on a magnetic particle membrane is present in a host magnetic bacterium, being covered with a coating membrane mainly comprised of proteins, and can be used as a carrier of physiologically active substances. The magnetic particle can be produced with the host magnetic bacterium mentioned above. For instance, there is a method for producing a magnetic particle expressing the present protein/peptide or the fusion protein/peptide on a magnetic particle membrane in a bacterium, by culturing magnetic bacteria being transformed by a recombinant plasmid comprising DNA that encodes part (at least a membrane-binding part) or whole of Mms 16, a membrane protein originally produced by binding to organic membrane, or DNA sequence wherein the DNA is fused with DNA that encodes a functional protein/peptide, and the promoter sequence having high transcriptional activity mentioned above. The magnetic particle expressing the present protein/peptide or the fusion protein/peptide on its magnetic particle membrane can be easily collected by using a magnet, after the above-mentioned magnetic bacteria proliferated by culture is homogenized or dissolved by known methods.

Specific example of a sandwich immunoassay method of the present invention is a method using a magnetic particle expressing a fusion protein, wherein an antibody as a functional protein and the present protein/peptide are bound, on its membrane surface, and a labeled antibody. As examples of a labeling substance, fluorescent materials such as FITC (fluorescein isothiocyanate) or tetramethylrhodamine isothiocyanate; radioisotopes such as ¹²⁵I, ³²P, ¹⁴C, ³⁵S or H; substances labeled with enzymes like alkaline phosphatase, peroxidase, β-galactosidase or phycoerythrin, or fluorescence emission proteins such as green fluorescent protein (GFP) are specifically exemplified. Examples of a method for measuring these substances include RIA method, ELISA method, fluorescent antibody technique, plaque method, spot method, hemagglutination reaction method and Ouchterony method. Sandwich immunoassay kit comprising a magnetic particle expressing a fusion protein, wherein an antibody as a functional protein and the present protein/peptide are bound, on its membrane surface, and a labeled antibody is also included in the present invention.

Any magnetic liposome can be used as a magnetic liposome of the present invention as long as it comprises a magnetic particle (magnetosome) expressing the present protein/peptide or the fusion protein/peptide on its magnetic particle membrane, and the magnetic liposome can be used for pharmaceuticals as a drug carrier. For example, it becomes possible to treat cancer and immunologic diseases by injecting a magnetic liposome, wherein a gene as a drug such as a remedy or an inhibitor for diseases, such as cancer and immunologic diseases, and the magnetic particle mentioned above are included, into a patient who suffers from cancer or immunologic diseases, and performing direction/fixation/release control of the magnetic liposome at an affected area by using an external magnetic field. Further, it is preferable to use magnet particles with high dispersibility for producing the magnetic liposome of the present inventon because the use of the magnet particles with high dispersibility will increase the inclusion rate of the drug mentioned above.

The present invention will be described more specifically with reference to examples, however, the scope of the present invention is not limited to these examples.

### EXAMPLE 1

### Bacterial Strains and Culturing Methods

Escherichia coli DH5a was used for cloning mms 16 gene and expressing Mms 16 protein. This E. coli DH5a was cultured at 37° C. in Luria medium added with ampicillin (50 μg/ml). A magnetic bacterium Magnetospirillum sp. AMB-1 (ATCC700264) was cultured at 26° C., in MSGM medium (pH 6.75) (J. Bacteriol. 140, 720-729, 1979), under an anaerobic condition (Appl. Microbiol. Biotechnol. 35, 651-655, 1991).

### EXAMPLE 2

### Cloning and Sequence Analysis of a Gene that Encodes the Mms 16 Protein of the Present Invention

A novel protein specific to a bacterial magnetic particle membrane, Mms 16 (molecular weight: 16 kDa), was identified by: fractionating cell homogenate of magnetic bacteria into 3 fractions of cell membrane, magnetic particle membrane, and cytoplasm, by the method previously described (Appl. Biochem. Biotech. 84-86, 441-446, 2000); separating each of the fractions by SDS-PAGE, and comparing the SDS-PAGE profiles of each protein. Subsequently, the proteins were separated by two-dimensional electrophoresis using TEP-2 unit (Shimadzu, Kyoto, Japan). After the separation, the electrophoretic gel was stained with Coomassie brilliant blue R-250. As a result, the Mms 16 had been separated into two spots. In order to determine each of amino acid sequences of N-terminal of the protein separated into two, the protein of the two spots were excised from the gel respectively, and blotted onto PVDF (polyvinylidene difluoride) membrane. The two proteins blotted onto the membrane were analyzed by Edaman degradation method using a gas-phase sequencer (PPSQ-10, Shimadzu, Kyoto, Japan), and amino acid sequences (58 residues) of N-terminal were determined, and, as a result, were found to be identical.

For the amplification of a base sequence that encodes the 1^{st} to the 44^{th} peptides from the N-terminal side of Mms 16 based on the above-mentioned amino acid sequence and codon frequency of Mag A (J. Biol. Chem. 270, 28392-28396, 1995), oligonucleotide primers (forward primer 1: 5′-CATAAGCAGACCGAGCAGTTCTTCGA-3′; Seq. ID No. 3, and reverse primer 1: 5′-TTGGCCTGGGTCAGGGCCTCGATGTT-3′; Seq. ID No. 4) were designed. For PCR, 1 to 10 ng of genomic DNA was used as a template. Thermal cycle program was as follows: a cycle of denaturation for 3 minutes at 94° C. at the beginning only, followed by thermal denaturation for 60 seconds at 94° C., annealing for 60 seconds at 65° C., and extension for 2 minutes at 72° C. was repeated 30 times, and extension for 15 minutes at 720 C was conducted lastly. As to PCR fragment (132 bp) wherein a base sequence encoding N-terminal peptide was amplified, the base sequence was determined by a cloning to pGEM (pGEM-T-easy Vector System; PROMEGA, WI, USA) and by using an automatic DNA sequencer DSQ-2000L (Shimadzu, Kyoto, Japan) or ABI PRISM 377 (Perkin-Elmer Co., Norfork).

After the determination of partial base sequence on the genome of mms 16 gene, primers for gene walking (primer of 5′ side: 5′-CGCTGGTTGGCGACGATGGTCTCGACATCC-3′; Seq. IDNO. 5, and primer of 3′ side: 5′-AAGTATCTGGGCGATTTCAAGGTTCC-3′; Seq. ID No. 6) were designed from a sequence inside a primer sequence in regions at both ends of 132 bp, and gene walking was conducted for the upstream and the downstream regions of regions, by using LA PCR in vitro Cloning Kit (TaKaRa, Shiga, Japan). As a result, a sequence of 1,235 bp (GenBank accession # AB051013) including ORF of 438 bp that encodes the protein Mms 16 was determined. Further, it was found that three promoter-like sequences and 14 palindromes were present in the region upstream of the initiating codon. As this Mms 16 protein exhibited the largest amount of expression on bacterial magnetic particle membranes, it is presumed that the protein is controlled by a high-expression promoter. In addition, as this protein does not express under aerobic culture (under the condition wherein magnetic particles are not produced), the presence of a presumable factor that regulates the condition under which magnetic particles are not produced, and regulation by the factor are presumed. A regulatory factor seems to bind to any one of the palindrome groups on the upstream of the promoter.

### EXAMPLE 3

### Analysis of Base Sequence and Amino Acid Sequence

DNA and amino acid sequences of Mms 16 protein that had been determined by the above-mentioned example were analyzed by using LASERGENE (DNASTAR INC. Madison, USA). Further, homology analysis was conducted by using GenBank, EMBL DNA databases. These database analyses have shown that the partial sequence on N-terminal region of Mms 16 protein of the present invention is homologous to GTP binding protein. In addition, the partial sequence on N-terminal region contains many GTP binding proteins for cell differentiation, transcriptional regulation, amino acid extension factor, signal recognition particle, signal transducer, etc., which are natively retained by cells, and homologous sequences. Genes that encode the above-mentioned GTP binding protein were non-specifically observed by PCR amplification mentioned in Example 2.

### EXAMPLE 4

### Expression and Isolation of Mms 16 Protein

In order to highly express Mms 16-hemagglutinin tag (HA) fusion protein in E. coli, PCR primers that amplify ORFs of a desired gene were designed. Reverse primer 2 was designed such that the sequence of hemagglutinin epitope tag (nine residues: YPYDVPDYA) was fused with C-terminal. PCR reaction was conducted with a forward primer 2 designed from EcoR I site (5′-GAATTCATGGCCGCCAAGCAGACTGAG-3′; Seq. ID No. 7) and a reverse primer 2 designed from Hind II site (5′-GGGAAGCTTGGCATAGTCGGGCACGTCATAGGGATACTTCTTGCCGGCCTTGGTGAA 3′; Seq. ID No. 8). Thermal cycle program was as follows: a cycle of denaturation for 3 minutes at 94° C. at the beginning only, followed by thermal denaturation for 60 seconds at 94° C., annealing for 60 seconds at 65° C., and extension for 2 minutes at 72° C. was repeated 30 times, and extension for 15 minutes at 72° C. was conducted lastly. Subsequently, PCR fragment encoding the amplified fusion protein was introduced into the downstream of Trc promoter of plasmid pTrc 99A (Amasham Pharmacia Biotech, Sweden), and pTrc 16HA was obtained.

E. coli DH5a was transformed with the plasmid pTrc 16HA mentioned above and Mms 16-HA fusion protein was expressed in E. coli in a medium added with 1 mM of IPTG. After the culture, E. coli was collected by centrifugation and resuspended in homogenization buffer (10 mM Tris-HCl; pH 8.0, 5 mM MgCl₂, 200 μg/ml phenylmethylsulphonyl fluoride), and homogenized by sonication. In addition, Western blotting with anti-HA antibodies has confirmed that fusion proteins locate in water-soluble fractions in the homogenization solution mentioned above, and it has been found that the water-soluble fractions can be easily used for purification process without considering a solubilizing condition. After anti-HA antibodies were bound to protein G sepharose beads (Amasham Pharmacia Biotech, Sweden), it was confirmed that the anti-HA antibodies were fixed to the protein G sepharose by SDS-PAGE and Western blotting, and cell homogenate was added to 50% (v/v) antibody-sepharose. The resultant solution was shaken for more than one hour at 4° C., and then its supernatant was removed by centrifugation (500×g), and the sepharose was washed 5 times with homogenization buffer. Precipitate was suspended in homogenization buffer to make up 50% (v/v), and the resultant suspension was used for the following Example as an immunoprecipitate.

### EXAMPLE 5

### SDS-PAGE and Western Blotting

Each cell homogenate and each immunoprecipitate of a wild type and a recombinant were respectively suspended in ½ amount of 3× sample buffer (0.1875 M Tris-HCl; pH 6.8, 15% 2-Mercaptoethanol, 6% SDS, 15% Sucrose, 0.006% Bromophenol blue) and denatured to prepare 4 kinds of sample for SDS-PAGE. For SDS-PAGE (Sodium dodecyl sulfate-polyacrylamide gel electrophoresis), 15% (w/v) acrylamide gel was used (Nature 227, 680-685, 1970), and 7 μg each of the 4 kinds of cell extract mentioned above was loaded onto each lane, proteins were stained with Coomassie brilliant blue R-250 or silver. Molecular weights were calculated from standard regression line with a low-molecular-weight calibration kit (Amasham Pharmacia Biotech, Sweden). After SDS-PAGE, gel was transferred to PVDF membrane by electroblotting and Western blotting was conducted. Mouse-derived anti-HA monoclonal antibody (1:5,000 dilution) was used for immunostaining, and alkaline phosphatase-labeled anti-mouse IgG antibody derived from goat was used as a secondary antibody.

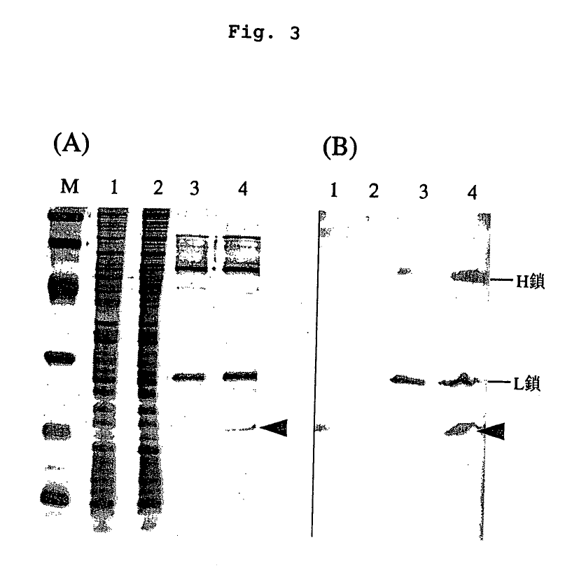

The results of the above-mentioned SDS-PAGE and Western blotting are shown in FIGS. 3A and 3B, respectively. In FIG. 3A, lane 1 was loaded with cell extract of a recombinant recombined by Mms 16-HA fusion gene, lane 2 was loaded with cell extract of awild type, lane 3 was loaded with aprotein collected from anti-HA antibody fixation beads which had been incubated with cell extract of a wild type and washed, and lane 4 was loaded with a protein collected from anti-HA antibody fixation beads which had been incubated with cell extract of a recombinant recombined by Mms 16-HA fusion gene and washed. These results of SDS-PAGE and Western blotting indicate that Mms 16-HA fusion protein is expressed in E. coli and can be purified by using anti-HA antibody fixation beads.

### EXAMPLE 6

### GTP Binding Ability

As Mms 16 of the present invention contained many GTP binding proteins and homologous sequences, the GTP binding ability of Mms 16 was investigated as follows. 1 μl of [γ-³⁵S] GTP (1 μM; 1,250 Ci/mmol) and cell homogenate were mixed in 20 μl binding buffer (50 mM Tris-HCl, 5 mM MgCl₂, 1 mM EDTA, 0.3% Tween 20), and kept still for one hour at 30° C. Then, the obtained reaction mixture was kept still on ice for 20 minutes, and ultraviolet bridging was performed between GTP and protein by using an ultraviolet lamp. Subsequently, ½ amount of 3× sample buffer was added to the reaction solution to denature the protein, and sepharose beads were precipitated by centrifugation, supernatant was loaded onto 15% (w/v) acrylamide gel. After SDS-PAGE, the gel was dried and wrapped in Saran Wrap, and detection was conducted by autoradiography.

As a result, in addition to the protein that binds to [γ-³⁵S] GTP that is observed also in a wild type (E. coli DH5α)), a band that is seen only in a transformant was observed in the cell homogenate, and it has been found that Mms 16 purified products apparently bind to [γ-³⁵S] GTP. In case where [α-³²P] GTP is used, [α-³²P] GTP binding type of GTPase is not observed because GDP also dissociates from GTPase when phosphoric acid in position γ dissociates from GTP binding type GTPase by hydrolysis activity. However, in case where [γ-³⁵S] GTP is used, it is possible to show the presence of GTPase because S is binding to 5′ of phosphoric acid in position γ, and therefore, hydrolysis is not caused by GTPase. Considering these results collectively, it is suggested that Mms 16 is a GTPase having GTP binding ability.

### EXAMPLE 7

### GTPase Activity

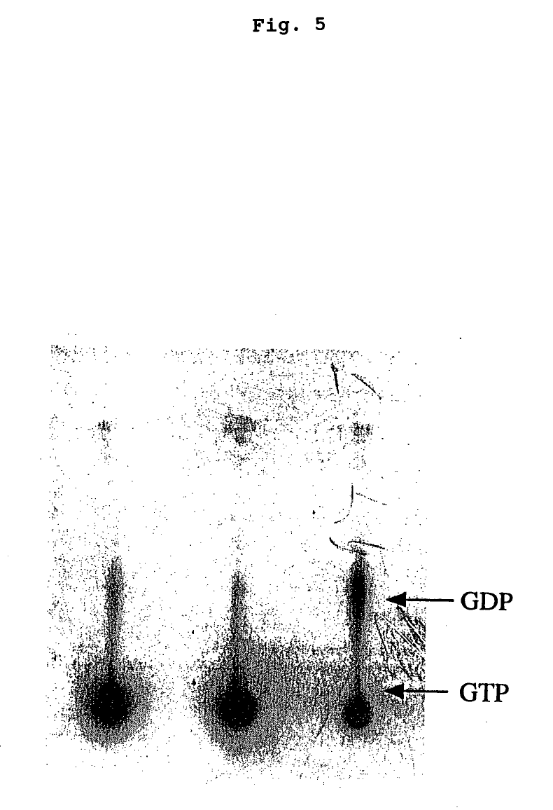

Based on the consideration of GTP binding result of Example 6, GTPase activity was examined. GTPase activity was measured as follows. Immunoprecipitate and [α-³²P] GTP or [γ-³²p] GTP were mixed together in 30 μl of binding buffer, and kept still for one hour at 30° C. Nucleotides in the reaction mixtures were developed with 0.75 M of KH₂PO₄ (pH 3.4) by using PEI cellulose TLC plastic sheet. The obtained thin-layer chromatography was wrapped with Saran Wrap, and made to contact with Fuji imaging plate (type BAS-IIIs; Fuji Photo Film, Kanagawa, Japan), and spots exposed to radioactivity were analyzed by bioimaging analyzer (BAS-1500, FUJIFILM). The results are shown in FIG. 5. In the figure, the left lane shows the result obtained by using binding buffer only, and the middle lane shows the result obtained by using proteins collected by a fusion protein purification procedure to cell extract of a wild type, and the right lane shows the result obtained by using Mms 16-hemagglutinin fusion protein purified by anti-HA antibody fixation beads. These results indicate that [α-³²P] GTP was hydrolyzed by Mms 16-HA fusion protein bound to the anti-HA antibody fixation sepharose complex and [α-³²P] GDP was produced, and that [³²P] Pi was produced from [γ-³²P] GTP, and therefore, it is shown that Mms 16 has GTPase activity.

### INDUSTRIAL APPLICABILITY

A foreign gene can be expressed in a cell by fusion of the foreign gene to Mms 16, a protein specific to a magnetic particle membrane of the present invention or its gene. Further, Mms 16, a protein specific to a magnetic particle membrane of the present invention can be used as a pharmaceutical or a diagnostic drug by being bound with a receptor, an antibody or the like.

What is claimed is:

1. DNA that encodes a protein described in the following (a) or (b):
(a) a protein comprising an amino acid sequence represented by Seq. ID No. 2,
(b) a protein comprising an amino acid sequence wherein one or a few amino acids are deficient, substituted or added in an amino acid sequence represented by Seq. ID No. 2, and having GTPase activity.

2. DNA that contains a base sequence represented by Seq. ID No. 1, its complementary sequence, and part or whole of these sequences.

3. DNA that encodes a protein hybridizing with DNA according to claim 2 under stringent conditions and having GTPase activity.

4. A promoter having high transcriptional activity that comprises part of DNA according to claim 2 or 3.

5. A protein comprising an amino acid sequence represented by Seq. ID No. 2.

6. A protein comprising an amino acid sequence wherein one or a few amino acids are deficient, substituted or added in an amino acid sequence represented by Seq. ID No. 2, and having GTPase activity.

7. A peptide comprising part of the protein according to claim 5 or 6, and having GTPase activity.

8. A fusion protein or a fusion peptide wherein the protein according to claim 5 or 6, or the peptide according to claim 7 is bound to a functional protein or a functional peptide.

9. The fusion protein or the fusion peptide according to claim 8, wherein the functional peptide is part of a functional protein, or a peptide tag.

10. The fusion protein or the fusion peptide according to claim 8 or 9, wherein the functional protein is one or more proteins selected from antigen, antibody, receptor, lectin, hormone, binding protein, enzyme, and marker protein.

11. An antibody that specifically binds to the protein according to claim 5 or 6, or the peptide according to claim 7 (claim 11),

12. The antibody according to claim 11, wherein the antibody is a monoclonal antibody.

13. A recombinant protein or a peptide that specifically binds to the antibody according to claim 11 or 12, and has GTPase activity.

14. A host cell comprising an expression system that can express the protein according to claim 5 or 6, the peptide according to claim 7, or the fusion protein or the fusion peptide according to any of claims 8 to 10.

15. The host cell according to claim 14, wherein the host cell is a host magnetic bacterium, Magnetospirillum sp.

16. The host cell according to claim 15, wherein the host magnetic bacterium, Magnetospirillum sp. is AMB-1 (FERM BP-5458, ATCC700264), MS-1 (IF015272, ATCC31632, DSM3856), MSR-1 (IF015272, DSM6361), RS-1 (FERM BP-13283), or MGT-1 (FERM P-16617).

17. A magnetic particle that can be obtained from the host magnetic cell according to claim 15 or 16.

18. A sandwich immunoassay method that uses an antibody being expressed on the magnetic particle according to claim 17, and a labeled antibody.

19. A magnetic liposome that comprises the magnetic particle according to claim 17.

20. A pharmaceutical composition that comprises the magnetic liposome according to claim 19 as an active component.


SEQUENCE LISTING (8 sequences)

SEQ ID NO: 1 - DNA, 1235 bp, Magnetospirillum sp.
  CDS (605)..(1042)
  misc_feature (1096)..(1096): n is a, c, g, or t
aaggsggtcatcgtgcgggccatgccmaataccccggcggcggtgcggcgcggcatcaccgtctgtgcgccggggaggccktgcccgtgtcgcccgtgagctttgccagtcgctgctggaagcggtgggcrgtgggctgggtcgatgacgagggcctgatggacgtggtcacscgtctccggctccgsccccgcctacatcttcctcctggccgaggccatggaggccgccggtctggcccaggggctgcccccgccctggccgagcgtctggcccgtgccaccgtggscggggccggcgaattgctgsgctgtccgccgaacccgccgagcaactgcgcaagaacgtcacctcgccgggcggcaccacagcggcggccctgtcggtgctgatgctgaaagccacggcattcccagcctgatgaccgaagcggtggctgctgccactcgccgaggacgggaacttgcgggctaggcgcttcgtcagaggcggtacctatttcgatattagatattgagttgcgtatgactccgtttgactcgaagccgcccccgcgtctatcttgctgcaccgcaacataagacccccgggttggaggaataacatggccgccaagcagactgagcagttctttgatttcgacgtcgccaagtatctgggcgatttcaaggttcccggcgtggatgtcgagaccatcgtcgccaaccagcgcaagaacatcgaagcgctgacccaggcgaacaagctggctttcgagggcctgcagaacgtggtcaagcgtcaggtcgagatcctgcgccagaccatggacgaggttgcccaggtctccaaggatttcgccgagcccggctcgccccagggcaaggccgccaagcaggccgagttcgccaaggatgccttcgagcgcgccctgrccaacgcccgtgagctggccgagatgatcgccaaggccaattccgaggctttcgacctgctgaacaagcgyttcacccagagcctggacgaggcccgcgaggtcttcaccaaggccggcaagaagtaagccttccgttcattcggaacgctgtcggcggccgctcctgaaaaggggcggctntttgattccggcctgaattgggcgctctaccgtccctcgatcagggccagaagcggcgcccattcggcggcgtaggacttggaacggcggtcattggcctcgatcaccgtcgagccggcggcggccaccgcggtataga

SEQ ID NO: 2 - protein, 145 aa, Magnetospirillum sp.
  misc_feature (104)..(104): Xaa can be any naturally occurring amino acid
MAAKQTEQFFDFDVAKYLGDFKVPGVDVETIVANQRKNIEALTQANKLAFEGLQNVVKRQVEILRQTMDEVAQVSKDFAEPGSPQGKAAKQAEFAKDAFERALXNARELAEMIAKANSEAFDLLNKXFTQSLDEAREVFTKAGKK

SEQ ID NO: 3 - DNA, 26 bp, Artificial Sequence
  Description of Artificial SequenceForward Primer 1
cataagcagaccgagcagttcttcga

SEQ ID NO: 4 - DNA, 26 bp, Artificial Sequence
  Description of Artificial SequenceReverse Primer 1
ttggcctgggtcagggcctcgatgtt

SEQ ID NO: 5 - DNA, 30 bp, Artificial Sequence
  Description of Artificial Sequence5′-primer
cgctggttggcgacgatggtctcgacatcc

SEQ ID NO: 6 - DNA, 26 bp, Artificial Sequence
  Description of Artificial Sequence3′-primer
aagtatctgggcgatttcaaggttcc

SEQ ID NO: 7 - DNA, 27 bp, Artificial Sequence
  Description of Artificial SequenceForward Primer 2
gaattcatggccgccaagcagactgag

SEQ ID NO: 8 - DNA, 57 bp, Artificial Sequence
  Description of Artificial SequenceReverse Primer 2
gggaagcttggcatagtcgggcacgtcatagggatacttcttgccggccttggtgaa